(19) United States
(12) Patent Application Publication
(10) Pub. No.: US 2007/0074346 A1
(43) Pub. Date: Apr. 5, 2007

(54) Application and method for blood control

(76) Inventor: Donald Wallace, Hanna City, IL (US)

Correspondence Address: HUSCH & EPPENBERGER, LLC, 190 CARONDELET PLAZA, SUITE 600, ST. LOUIS, MO, 63105-3441, US

(21) Appl. No.: 11/201,040

(22) Filed: Aug. 10, 2005

(51) Int. Cl. A47B 71/00; A61F 13/00; A61F 13/15; A61F 15/00

(52) U.S. Cl. 5/600; 602/56; 604/393

(57) ABSTRACT

A medical procedure fluid control apparatus includes a procedure bed, and at least one wing. The wing is along at least one longitudinal edge of the procedure bed, and is moveable to a plane non-parallel with a plane of the procedure bed. The apparatus has at least one absorbent portion being on a surface of the procedure bed or the wing. The at least one absorbent portion is disposed to face the patient. At least one adhesive portion of the fluid control apparatus is also on a surface of the procedure bed or said wing disposed to face patient.

### CROSS-REFERENCE TO RELATED APPLICATIONS

None.

### BACKGROUND OF THE INVENTION

1. Field of the Invention

This invention is in the medical supply field, in particular in the field of devices for phlebotomy procedures, intravenous catheter insertions and extremity wound treatment.

2. Related Art

Speed, accuracy and cleanliness are always valued in medical treatment. This is particularly true in medical procedures involving the management of blood, including intravenous catheter placement and trauma treatment. The advent of HIV, hepatitis C and other blood borne pathogens increases the importance of the need for accurate, controlled, efficient and economical tools for executing these medical procedures.

In particular when a trauma patient enters an emergency room with an extremity wound, or more commonly when a nurse or other health care provider places an intravenous catheter, or when blood samples are drawn, there is a need for containing, controlling and absorbing any spillage of blood during the procedure. Furthermore, there is a need to provide economic, easy to use equipment that is preferably disposable and may also may preferably have multiple uses.

Currently, emergency rooms and phlebotomists have little more than towels or gauze pads or bandages for absorbing any blood spilled during a blood sampling, IV catheter placement or treatment of some trauma to extremities. There is a need in the art for a blood control device tailored to these circumstances.

### SUMMARY OF THE INVENTION

The invention herein is an absorbent procedure bed for use upon extremities. The bed may be flexible or semi-rigid. An absorbent layer is designed to face the patient and come into contact with the extremity upon which the procedure is to be performed. For further advantageous control, adhesive is provided at selected locations on an upper surface of the absorbent sheet such that the adhesive may be disposed to adhere to the patient's skin at selected locations. Adhesive areas may be interspersed with absorbent areas.

In another aspect of the present invention, the procedure bed is provided with additional tools useful to the phlebotomist or other medical practitioner. These may include a pocket or series of pockets or bandoleers for placement of blood sample vials or other tools. It may further include additional strips having attachments that may be adhesive, hook and eye attachments or otherwise for holding the procedure bed to the extremity and/or for holding an intravenous catheter in a desirable, practitioner selected position. It may further include a strip or sleeve at a proximally disposed edge of the procedure bed into which a tourniquet may be placed. Finally, a sleeve, slot or other receptacle may be disposed on a bottom portion of the procedure bed for insertion of stiffeners such as battens or boards. Such stiffeners would advantageously keep an extremity rigid for trauma procedures involving bone fractures or otherwise requiring a splint.

The procedure bed may be entirely flexible or, in the alternative, may be semi-rigid and formable such that wings of the procedure bed may be elevated on either side of the extremity for further control of bleeding, or spillage.

The absorbent sheet and procedure bed of the present invention may further advantageously be constructed and arranged to be maintained in a sterile individual packaging, or sterile multiple unit packaging, such as in a roll, or nonsterile multiple packaging as in stacks or in rolls.

Another aspect of the present invention is a provision for control of fingers and hands in a pediatric application. A slot, sleeve, adhesive band or adhesive bed may be disposed at a distal end of the procedure bed in order that a small child's fingers, hands or foot may be more readily held stable during a procedure.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of the procedure that of the present invention.

FIG. 2 is a cutaway side view of the procedure in use.

FIG. 3 is a top view of showing adhesive and absorbent areas.

FIG. 4 is a perspective view showing adhesive and absorbent areas.

FIG. 5 is an end view showing sleeves for stiffening battens.

FIG. 6 is a top view of an alternative embodiment.

FIG. 7 is a side view of an alternative embodiment.

FIG. 8 is a perspective view of an alternative embodiment including a temporary splint.

### DETAILED DESCRIPTION OF THE PREFERRED EMBODIMENTS

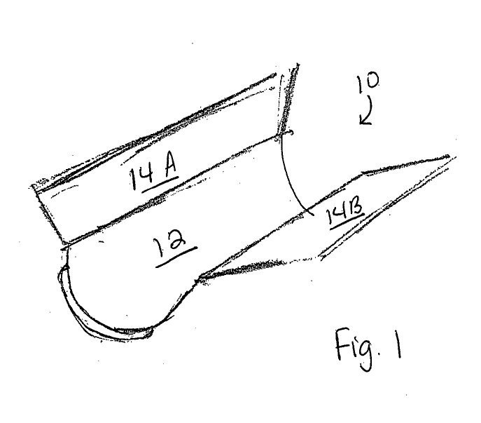

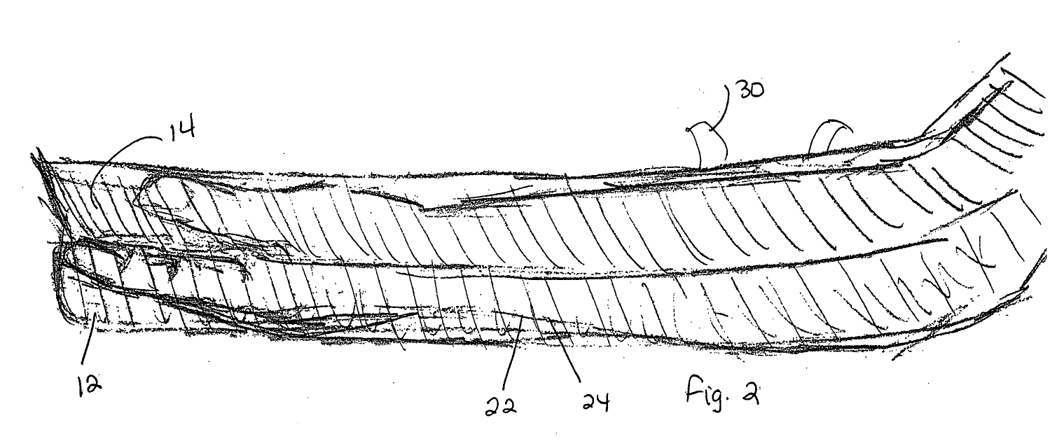

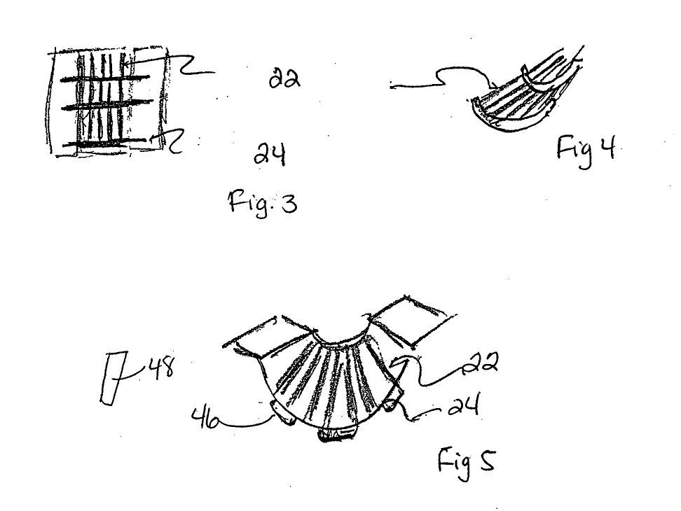

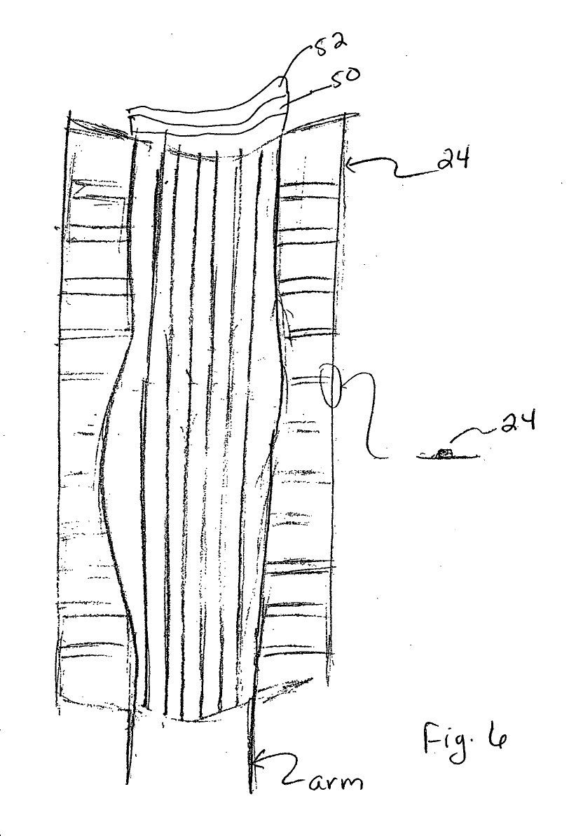

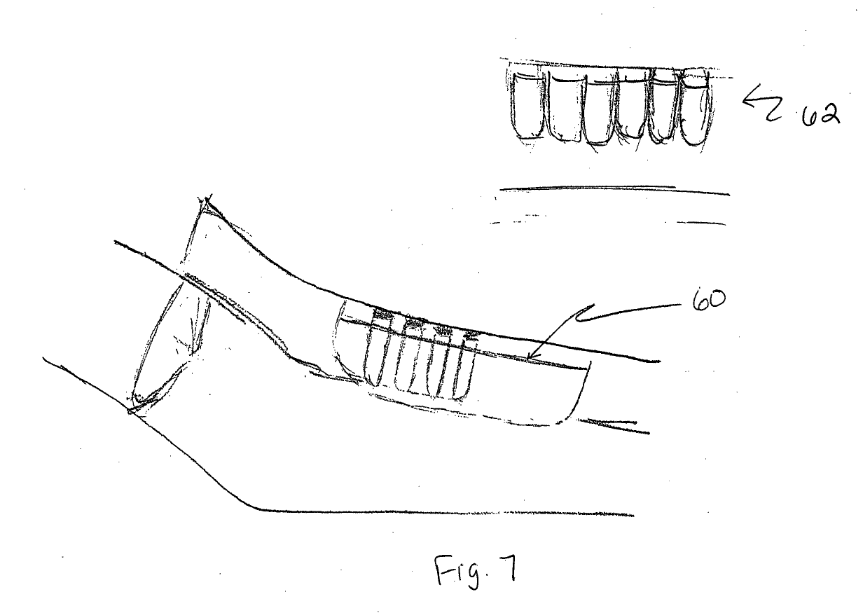

The following description of the preferred embodiment(s) is merely exemplary in nature and is in no way intended to limit the invention, its application, or uses. Referring now to the drawings where like numbers indicate like elements, FIG. 1 is a perspective view of the procedure bed of the present invention. Procedure bed assembly 10 is comprised of a procedure bed 12 flanked by wings 14A and 14B oriented parallel with the longitudinal axis of the procedure bed 12 and on the sides of the procedure bed. The procedure bed 12, wings 14 individually, together or in any combination may be either fully flexible or semi-rigid. A curvature may be obtained by constructing the procedure bed assembly with flexible plastic panels, or by constructing the procedure bed assembly with sleeves or rear adhesive portions for placement of stiffeners. In the embodiment depicted in FIG. 1, a sleeve 16 is provided underneath the procedure bed 12 for insertion of a longitudinal stabilizer 18.

FIG. 2 shows the procedure bed assembly 10 in use. A patient's arm is depicted in the side view. Procedure bed 12 is placed on a procedure table (not shown) and the patient's arm is placed on the procedure bed 12. In those embodiments having stiffening components, the patient's extremity is placed in the trough formed by the procedure bed 12. Wings 14 are disposed to angle upwards and away from an upper aspect of the patient's extremity upon which a procedure is to be formed. In this manner, a bleeding extremity, an extremity that needs to be washed or an otherwise clean extremity that may be subject to spillage or bleeding during a procedure is effectively surrounded on the bottom and sides in order that blood, lavage or other fluids are controlled and retained in the area of the procedure bed there to be absorbed.

Both the procedure bed 12 and wings 14 in the embodiment depicted in FIG. 2 are shown having alternate transverse strips. These strips represent a disposition of adhesive 22 and absorbent material 24, which may be alternating in the embodiment depicted in FIG. 2. Construction of the procedure bed assembly may be by starting with an absorbent substrate such as gauze, absorbent polymers or other absorbent materials with adhesive strips attached on top of the absorbent substrate in order that an adhesive surface face the patient's extremity. In this manner, the procedure bed assembly is held in its desired, advantageous position while an absorbent surface is maintained proximal to the patient's skin and, in some cases to a bleeding wound. Additionally, straps or tabs 30 may be placed on the wings as depicted. These straps or wings may be constructed with more adhesive material, hooks and eyes, hook and eye pads or other means for attachment of tabs. These tabs may be used to further stabilize and maintain the procedure bed in the desired position. These strips may further be used to stabilize and maintain an intravenous catheter line in a desired position. In most cases this position of an IV line is parallel with the longitudinal axis of the extremity and immediately adjacent to skin. In the prior this position was maintained only with simple adhesive tape or not at all.

FIGS. 3 and 4 depict a top view and a perspective view, respectively, of an alternative design configuration for adhesive and absorbent portions of an upper surface of the procedure bed assembly. Adhesive strips 22 may be arranged perpendicular to absorbent areas 24. It is within the scope of the present invention that any geometric pattern may be used to provided that there is an absorbent portion and adhesive portion of an upper surface of the procedure bed facing the patient's extremity. Moreover, adhesive areas are provided with a non-adhesive cover which is removed immediately before use exposing the adhesive surface to the patient's skin. The procedure bed assembly may be constructed and arranged so that different adhesive portions are provided with separate covers. In this fashion, a healthcare practitioner may selectively expose the adhesive on different adhesive areas in order to tailor the use of the procedure bed assembly to the patient need at hand. For example, the cover may be left on an adhesive strip to be placed immediately proximate a wound so that the device does not stick to the wound.

FIG. 5 shows an alternate embodiment having multiple sleeves 46 for receiving and maintaining stabilizing battens 48. FIG. 5 also shows longitudinal alternating adhesive and absorbent areas, again with separate covers being provided for the adhesive areas. FIG. 6 is a top view of another alternative embodiment of the present invention. The patient's arm is shown over the procedure bed. Shown in phantom on the underside of the patient's arm are the longitudinal alternating adhesive and absorbent strips on the procedure bed portion 12. The wings are disposed with alternating with a series of absorbent ribs oriented in a transverse direction. Such transverse absorbent areas may be useful in a situation where the extremity is not parallel to the ground. As such circumstances, blood, water or lavage fluid would flow downwards. The raised ribs of the depicted embodiment would aid in controlling spillage of such fluid.

Also depicted in FIG. 6 is a cuff 50 having either a permanent transverse through hole for receiving a tourniquet, or, alternatively, a flexible wraparound portion with a transverse adhesive or other attachment strip 52. Thus, a user may place a tourniquet device, such as a rubber hose or latex strap (not shown) over the cuff portion thereafter to be wrapped around by the cuff and held secure in the user selected position by the adhesive portion 52.

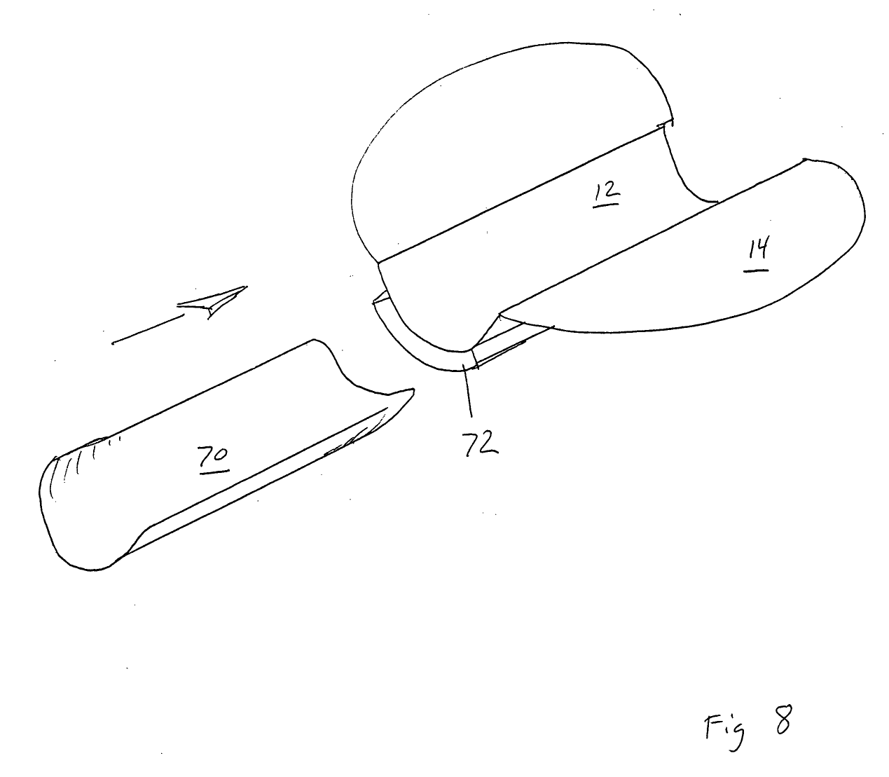

FIG. 7 depicts a further alternative embodiment. Frequently phlebotomists and the like are charged with taking blood samples. This procedure is executed by piercing a vein, positioning a catheter in the vein and placing a socket device over the catheter. The socket device receives serial blood vials for taking multiple samples separately. The vials are placed one by one in the socket. Typically the phlebotomist places the empty bottles in any fashion on a procedure surface next to a patient's extremity before and after they are filled. A more satisfactory control of these vials is achieved by providing a temporary storage space for them on the procedure bed assembly. Hence, pockets 60 along the wings or bandoliers 62 are provided for this use.

In another aspect of the present invention, the stiffeners may be rigid enough to provide a splinting effect for any fractures of the extremity being treated. In on-site emergency treatment and/or transportation, it is often beneficial to provide stability to an extremity wound that includes a fracture. Upon more thorough treatment, as for example at the emergency room of a hospital, complex wound treatment priorities may favor treating a badly bleeding vessel or a compromised nerve before reducing a fracture. While such prioritized treatment is proceeding, it is useful to maintain the extremity in a rigidly maintained position, as with a temporary splint. Accordingly, in FIG. 8, an embodiment of the invention is depicted wherein the procedure bed external surface includes a single large sleeve 72 dimensioned to receive a stiffening device 70 that is wider than the battens depicted in FIG. 5. With either the battens of FIG. 5 or the stiffener of FIG. 8, these devices may be rigid enough to provide a splinting effect for fractured extremities. In each case, the batten or stiffener may have some curvature around a radius substantially parallel with the long axis of the extremity being treated, and/or may be made of any appropriate material, including without limitation plastic or metal.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A medical procedure fluid control apparatus comprising:
a procedure bed;
at least one wing, said at least one wing being along at least one longitudinal edge of said procedure bed, said at least one wing being moveable to a plane non-parallel with a plane of said procedure bed;
at least one absorbent portion of said fluid control apparatus, said at least one absorbent portion being on a surface of said procedure bed or said wing disposed to face a patient; and
at least one adhesive portion of said fluid control apparatus, said at least one adhesive portion being on a surface of said procedure bed or said wing disposed to face a patient.

2. The apparatus of claim 1 further comprising a second wing, said second wing being disposed on an opposite side of said procedure bed from said at least one wing.

3. The apparatus of claim 1 further comprising a crease between said procedure bed and said at least one wing.

4. The apparatus of claim 1 further comprising a sleeve, said sleeve being adapted to receive a batten and said sleeve being disposed on a side of said procedure bed or said at least one wing facing away from a patient.

5. The apparatus of claim 1 having a plurality of adhesive portions and a plurality of absorbent portions.

6. The apparatus of claim 5, wherein said adhesive portions and said absorbent portions are alternating.

7. The apparatus of claim 1 further including a splint.

8. The apparatus of claim 1 further including a transverse throughhole on a proximal end of said apparatus, said throughhole being dimensioned to receive and retain a tourniquet.

9. The apparatus of claim 1 further comprising a tourniquet, said tourniquet being attached to a proximal end of said apparatus.

10. The apparatus of claim 1 further comprising at least one pocket, said pocket being attached to said apparatus and said pocket being dimensioned to receive and retain a vial.

11. The apparatus of claim 1 further comprising at least one adhesive tab for retaining said wings in a user selected position around a patient's extremity.

12. The apparatus of claim 1 wherein said apparatus is curved.

13. The apparatus of claim 1 wherein said procedure bed is concave towards a patient.

14. The method of making a medical procedure fluid control apparatus comprising:
fabricating a procedure bed;
attaching at least one wing along at least one longitudinal edge of said procedure bed, said at least one wing being moveable to a plane non-parallel with a plane of said procedure bed;
facing towards a patient at least one absorbent portion, said at least one absorbent portion being on at least one of said procedure bed or said wing; and
facing at least one adhesive portion of said fluid control apparatus towards a patient, said at least one adhesive portion being on one of said procedure bed or said wing.

15. The method of claim 14 further comprising attaching a second wing, said second wing being disposed on an opposite side of said procedure bed from said at least one wing.

16. The method of claim 14 further comprising creating a crease between said procedure bed and said at least one wing.

17. The method of claim 14 further comprising adding at least one sleeve, said sleeve being adapted to receive a batten and said sleeve being disposed on a side of said procedure bed or said at least one wing facing away from a patient.

18. The method of claim 14 a plurality of adhesive portions and further comprising alternating a plurality of absorbent portions.

19. The method of claim 14, further comprising fixing at least one pocket to said apparatus.

20. The method of claim 17 further including a splint, said splint being insertable into said sleeve.
